(19) United States
(12) Patent Application Publication
(10) Pub. No.: US 2007/0032677 A1
(43) Pub. Date: Feb. 8, 2007

(54) Powdery, water-soluble cationic polymer compositions, method for the production and use thereof

(75) Inventors: Gregor Herth, Trostberg (DE); Bernd Kubiak, Krefeld (DE); Norbert Steiner, Alzenau (DE); Eric Benghozi, Rietmattstrasse (CH)

Correspondence Address: C. IRVIN MCCLELLAND;OBLON, SPIVAK, MCCLELLAND, MAIER & NEUSTADT, P.C., 1940 DUKE STREET, ALEXANDRIA, VA, 22314, US

(73) Assignee: Stockhausen GmbH, Krefeld (DE)

(21) Appl. No.: 10/567,909

(22) PCT Filed: May 28, 2004
(86) PCT No.: PCT/EP04/05810

§ 371 Date: Oct. 6, 2006

(51) Int. Cl. C07C 69/52

(52) U.S. Cl. 560/205

(57) ABSTRACT

The invention relates to powdery, water-soluble cationic polymer compositions comprising at least two cationic polymer compositions comprising at least two cationic polymers having a different molecular weight. A first cationic polymer is formed in the presence of a second cationic polymer from monomer constituents in an aqueous solution according to the aqueous gel polymerization method. The invention also relates to the use of products in solid/liquid separation.

The present invention relates to powdery, water-soluble, cationic polymers composed of at least two different cationic polymer components, which are different in terms of cationic components and molecular weight, as well to a method for production of same and to the use of the polymer products for solid-liquid separation, for example as a retention aid in paper manufacture, and in sludge dewatering/wastewater purification.

In the practice of solid-liquid separation, the object is to achieve, by addition of flocculating auxiliaries, the best possible result in terms of the parameters dry substance of the solid and clarity of the filtrate, or in other words to bring about the most complete separation possible of solid from the liquid phase. Sludge dewatering on a chamber-type filter press can be regarded as an example of the importance of these parameters. Since the dried sludge must be transported and often put to beneficial use by thermal processing, the highest possible content of solid (dry-substance content) is desired. In addition, the separated filtrate must be delivered to disposal. The quality and simplicity of such disposal increase as the clarity of the filtrate increases, or in other words as the content of unflocculated solids remaining in the filtrate becomes lower. In such a case the filtrate can be discharged directly from a clarifying plant to the environment, and does not have to pass through the clarifying plant once again. Occasionally a flocculating auxiliary produces a flocculated sludge with high solid content but unsatisfactory clarification of the supernatant. The situation may be the reverse for another flocculating agent.

Flocculating auxiliaries are produced in the form of powdery granules or water-in-water or water-in-oil emulsions, and prior to their use are added in dilute aqueous solutions to the medium to be flocculated. Powdery granules are preferred, since they can be transported more inexpensively by virtue of their almost anhydrous condition and, as in the water-in-oil emulsions, do not contain any oil or solvent constituents that are insoluble in water.

It has been found in practice that the combination of two flocculating auxiliaries often yields better overall results than the use of a single flocculating auxiliary. For example, DE-OS (German Unexamined Application) 1642795 and EP 346159 A1 describe the successive addition of different polymeric flocculating agents.

Mixtures of powdery granules are described in the prior art, for example in WO 99/50188, wherein powders of two oppositely charged flocculating auxiliaries are united in a common solution. By virtue of different dissolution behavior of the two polymer powders, solution products of irregular composition can already be formed during the dissolution operation.

The use of dry powder mixtures of different polymers in flocculation processes can lead to faulty proportioning as a result of phase-separation phenomena. From EP 262945 A2 there are known cationic flocculation auxiliaries composed of two different polymer components and methods for production of same. They are not obtained by mixing the polymer components together but are formed by polymerization of cationic monomers to a high molecular weight cationic polymer component (flocculent) in the presence of a low molecular weight cationic polymer component (coagulant). During this polymerization reaction, the polymer added first can undergo graft reactions. Because of their incompatibility with the flocculent, which is based on acrylate monomers, the following coagulant polymers are preferably used: polymers of allyl monomers, especially poly-DADMAC and amine-epichlorohydrin polymers (page 4, line 40 et seq.). The ratio of coagulant to the high molecular weight polyelectrolyte component is specified as 10:1 to 1:2, preferably 5:1 to 1:1.5 (page 3, lines 48-49), or in other words the proportion of coagulant in the polymer mixture is 83 to 40 wt % in the preferred embodiment. The high proportions of coagulant cause viscosity problems in the production of polymerization solutions. The properties of the disclosed flocculating agents do not satisfy the requirements of speed and effectiveness imposed on technical flocculation processes.

The object of the present invention was to provide powdery cationic flocculation auxiliaries that are improved compared with the prior art and that are composed of a low molecular weight polymer constituent and a high molecular weight polymer constituent. Another object is to specify a production method by which the two polymer components can be united with one another without substantial restrictions and the reaction products can be further processed without substantial restrictions, and wherein an intrinsically uniform and readily soluble polymer powder is formed.

The object is achieved by a water-soluble, cationic polymer composition that contains at least two cationic polymers differing in molecular weight, wherein a first cationic polymer is formed by radical polymerization of its monomer constituents in the presence of a second cationic polymer in aqueous solution, characterized in that
the cationic structural units of the first and second polymer are of corresponding type,
the polymerization of the first cationic polymer takes place in an aqueous solution of the second cationic polymer according to the method of adiabatic gel polymerization.

In an advantageous embodiment, the polymer composition is formed by a ratio of the second cationic polymer to the first cationic polymer of 0.01:10 to 1:3, preferably 0.1:10 to <1:4 and particularly preferably 0.2:10 to <1:10.

According to the invention, the two cationic polymers have the same cationic components, which, compared with the statement of EP 262945 A2, according to which good results are to be achieved only by different cationic components, is to be regarded as surprising.

The first cationic polymer is a copolymer of cationic and nonionic monomers; in contrast, the second cationic polymer can be both a copolymer and a homopolymer.

Examples of suitable cationic monomer components are cationized esters of (meth)acrylic acid, such as dimethylaminoethyl(meth)acrylate, diethylaminoethyl(meth)acrylate, diethylaminopropyl(meth)acrylate, dimethylaminopropyl(meth)acrylate, diethylaminopropyl(meth)acrylate, dimethylaminobutyl(meth)acrylate, diethylaminobutyl(meth)acrylate, cationized amides of (meth)acrylic acid, such as dimethylaminoethyl(meth)acrylamide, diethylaminoethyl(meth)acrylamide, diethylaminopropyl(meth)acrylamide, dimethylaminopropyl(meth)acrylamide, diethylaminopropyl(meth)acrylamide, dimethylaminobutyl(meth)acrylamide, diethylaminobutyl(meth)acrylamide, cationized N-alkylmonoamides and diamides with alkyl groups containing 1 to 6 C atoms, such as N-methyl(meth)acrylamide, N,N-dimethylacrylamide, N-ethyl(meth)acrylamide, N-propyl(meth)acrylamide, tert-butyl(meth)acrylamide, cationized N-vinylimidazoles as well as substituted N-vinylimidazoles, such as N-vinyl-2-methylimidazole, N-vinyl-4-methylimidazole, N-vinyl-5-methylimidazole, N-vinyl-2-ethylimidazole and cationized N-vinylimidazolines, such as vinylimidazoline, N-vinyl-2-methylimidazoline and N-vinyl-2-ethylimidazoline.

The basic monomers are used in the form neutralized with mineral acids or organic acids or in quaternized form, in which case quaternization is preferably effected with dimethyl sulfate, diethyl sulfate, methyl chloride, ethyl chloride or benzyl chloride. In a preferred embodiment, the monomers quaternized with methyl chloride or benzyl chloride are used.

Preferred cationic monomer components are the cationized esters and amides of (meth)acrylic acid, in each case containing a quaternized N atom. Particularly preferably there are used quaternized dimethylaminopropylacrylamide and quaternized dimethylaminoethyl acrylate.

Examples of suitable nonionic monomer components, which are preferably water-soluble, are acrylamide, methacrylamide, acrylonitrile, methacrylonitrile, N,N-dimethylacrylamide, vinylpyridine, vinyl acetate, hydroxy-group-containing esters of polymerizable acids the hydroxyethyl and hydroxypropyl esters of acrylic acid and methacrylic acid, further amino-group-containing esters and amides of polymerizable acids, such as the dialkylamino esters, for example dimethylamino and diethylamino esters of acrylic acid and methacrylic acid, a specific example being dimethylaminoethyl acrylate, as well as the corresponding amides, such as dimethylaminopropylacrylamide. Preferably acrylamide is used as the nonionic monomer component. Monomers having limited solubility in water are used only to the extent that they do not impair the water solubility of the resulting copolymer.

The first cationic polymer is a high molecular weight polymer. Its average molecular weight Mw is higher than 1 million, preferably higher than 3 million. The molecular weight of the first cationic polymer is higher than that of the second cationic polymer. The high molecular weight of the first cationic polymer improves the effect of the inventive polymer composition in the flocculation process.

The charge density of the first cationic polymer can be freely selected in principle, and must be matched to the respective application. In one advantageous embodiment, the first cationic polymer is formed from 20 to 90 wt %, preferably 40 to 80 wt % of cationic monomers.

In one advantageous embodiment, the second cationic polymer is formed from 70 to 100 wt %, preferably from 75 to 100 wt % and particularly preferably from 100 wt % of cationic monomers.

The second cationic polymer has lower molecular weight than the first cationic polymer. Its average molecular weight is lower than 1 million, preferably between 50,000 and 700,000 and particularly preferably between 100,000 and 500,000.

In a further advantageous embodiment, the first cationic polymer has a lower cationic charge density than the second cationic polymer.

The inventive water-soluble, cationic polymer compositions are produced by the method of adiabatic gel polymerization, wherein a first cationic polymer is formed by radical polymerization of its monomer constituents in aqueous solution in the presence of a second cationic polymer.

For the reaction, an aqueous solution of cationic and if necessary nonionic monomers and the second cationic polymer is first prepared, the start temperature for the polymerization is adjusted to a range of −10° C. to 25° C., and oxygen is purged from the solution by an inert gas. The exothermic polymerization reaction of the monomers is started by addition of a polymerization initiator, and heating of the polymerization mixture takes place with formation of a polymer gel. After the temperature maximum has been reached, the solid polymer gel being formed can be further processed immediately or else after a holding time. Preferably the polymer gel will be further processed immediately after the maximum temperature has been reached.

The aqueous mixture of monomers and the second cationic polymer is usually prepared in a concentration of 10 to 60 wt %, preferably 15 to 50 wt % and particularly preferably 25 to 45 wt %.

In a preferred embodiment, the solution obtained during polymerization of the second cationic polymer is used directly for production of the inventive products.

The start temperature for the polymerization reaction is adjusted to a range of −10° C. to 25° C., preferably to a range of 0° C. to 15° C. Higher start temperatures lead to polymer gels which are too soft to be further processed in the subsequent size-reduction and drying processes.

The polymerization of the first cationic polymer is performed as an adiabatic polymerization, and it can be started either with a redox system or with a photoinitiator. Moreover, a combination of the two starting options is possible.

The redox initiator system comprises at least two components: An organic or inorganic oxidizing agent and an organic or inorganic reducing agent. For this purpose there are often used compounds with peroxide units, examples being inorganic peroxides such as alkali metal and ammonium persulfate, alkali metal and ammonium perphosphates, hydrogen peroxide and its salts (sodium peroxide, barium peroxide) or organic peroxides such as benzoyl peroxide, butyl hydroperoxide or per acids such as peracetic acid. Besides those, however, other oxidizing agents can also be used, such as potassium permanganate, sodium and potassium chlorate, potassium dichromate, etc. As reducing agents there can be used sulfur-containing compounds such as sulfites, thiosulfates, sulfinic acid, organic thiols (ethylmercaptan, 2-hydroxyethanethiol, 2-mercaptoethylammonium chloride, thioglycolic acid) and others. In addition, ascorbic acid and low-valency metal salts are possible [copper (I); manganese (II); iron (II)]. It is also entirely possible to use phosphorus compounds, such as sodium hypophosphite. In the case of photopolymerization, the reaction is preferably started with UV light, which causes decomposition of the initiator. As examples, benzoin and benzoin derivatives, such as benzoin ether, benzil and its derivatives, such as benzil ketals, acryldiazonium salts, azo initiators such as 2,2′-azobis(isobutyronitrile), 2,2′-azobis(2-amidinopropane) hydrochloride or acetophenone derivatives can be used as initiators. The quantity of the oxidizing and reducing components ranges between 0.00005 and 0.5 wt %, preferably from 0.001 to 0.1 wt %, and that of photoinitiators ranges between 0.001 and 0.1 wt %, preferably 0.002 to 0.05 wt %, relative to the monomer solution.

The polymerization is carried out in aqueous solution, in batches in a polymerization vessel or continuously on an endless belt, as is described, for example, in DE 3544770. This specification is herewith made part of the disclosure by reference. The process is carried out at atmospheric pressure without external supply of heat, a maximum final temperature of 50 to 150° C., depending on the concentration of polymerizable substance, being reached due to the heat of polymerization.

According to this inventive polymerization procedure, there are obtained polymers with decisively better product properties than were measured for products according to EP 262945, which products were synthesized by isothermal polymerization.

After the end of polymerization, the polymer existing as a gel is subjected to size reduction in standard industrial apparatus. The ratio of the second to the first cationic polymer is decisive for further processing of the polymer gel. If the ratio exceeds the value of 0.01:10 to 1:4, there are formed very soft gels, which immediately coalesce once again after size reduction and make drying on the industrial scale almost impossible. Polymers with cationic monomer proportions of greater than 60 wt % are particularly critical as regards further processing. In those cases, it has often proved effective to adjust the ratio of the first to the second cationic polymer to 0.2:10 to <1:10.

After size reduction, the gel is dried in batches in a circulating-air drying oven at 70° C. to 150° C., preferably at 80° C. to 120° C. and particularly preferably at 90° C. to 110° C. In the continuous version, drying takes place in the same temperature ranges, for example on a belt dryer or in a fluidized-bed dryer. After drying, the product preferably has a moisture content of less than or equal to 12%, and particularly preferably of less than or equal to 10%.

After drying, the product is ground to the desired particle-size fraction. In order to achieve rapid dissolution of the product, at least 90 wt % of the product must have a size of smaller than 2.0 mm, and preferably 90 wt % must have a size of smaller than 1.5 mm. Fine fractions smaller than 0.1 mm should amount to less than 10 wt %, preferably less than 5 wt %.

The inventive polymers are suitable as flocculation auxiliaries in the course of solid/liquid separation. In particular, they can be used suitably for purification of wastewater and for conditioning of potable water. Above and beyond this, they can be advantageously used as retention auxiliaries in flocculation processes during paper manufacture.

The invention will be explained hereinafter on the basis of examples. These explanations are provided exclusively by way of example and do not limit the general inventive ideas.

### EXAMPLES

Determination of the Viscosity of the Polymer

The viscosities were determined with a Brookfield viscometer on a 0.5 wt % solution in 10 wt % NaCl solution. The dissolution time was one hour.

The following abbreviations are used:
ABAH: 2,2′-azobis(2-amidinopropane)hydrochloride
DIMAPA-quat: 3-dimethylammoniumpropyl(meth)acrylamide, which has been quaternized with methyl chloride
ADAME-quat: 2-dimethylammoniummethyl(meth)acrylate, which has been quaternized with methyl chloride
DADMAC diallyldimethylammonium chloride
   Second Cationic Polymer

The second cationic polymers used in the examples are solution polymers of DIMAPA-quat, which were produced with various polymer contents and various molecular weights (Mw according to GPC). The properties of these products are listed in more detail in the table:

| | | Polymer | Molecular |
|---|---|---|---|
| | Type | content | weight |
| K1 | Poly-DADMAC | 40% | 300,000 |
| K2 | Poly-DIMAPA- | 25% | 1,000,000 |
| | quat | | |
| K3 | Poly-DIMAPA- | 40% | 100,000 |
| | quat | | |
| K4 | Poly-DIMAPA- | 25% | 500,000 |
| | quat | | |

Determination of the Dewatering Effect by the Screen-Test Method

This test method is adapted to dewatering methods used in industry, namely continuous pressure filtration by means of filter presses or centrifugal dewatering in centrifuges.

By means of this method, organic cationic polymers are usually tested with regard to their suitability for conditioning and dewatering of communal or industrial sludges.

Using the flocculation-auxiliary solution to be tested, the sludge is conditioned under constant conditions (depending on the existing dewatering equipment). After conditioning, the sludge sample is filtered (=dewatered) on a metal screen (200 μm mesh openings). The dewatering time (t_{E}) for a predefined volume of filtrate is measured, and the clarity of the collected filtrate is evaluated in a clarity wedge (optically).

| | |
|---|---|
| Clarity: “0” = | no clarification |
| Clarity: “46” = | best clarification |

Inventive Polymers:

The inventive polymers are produced by the method of gel polymerization.

Polymer 1

390.0 g of 50 wt % aqueous acrylamide solution was first placed in a polymerization vessel and 93.8 g of water as well as 210 mg of Versenex 80 was mixed in. After the addition of 325.0 g of 60 wt % DIMAPA-quat and 140.0 g of the 25 wt % solution of K2, the pH was adjusted to 5.0 with 4.0 g of 50 wt % sulfuric acid and the mixture was cooled to 0° C. and purged with nitrogen. After the addition of 0.45 g of ABAH (2,2′-azobis(2-methylpropionamidine)dihydrochloride), the polymerization was started with UV light. Within 25 minutes, the polymerization went from 0° C. to 80° C. The polymer was subjected to size reduction with a meat grinder and dried at 100° C. for 90 minutes. The product was ground to a particle-size fraction of 90 to 1400 μm.

Polymer 2

The synthesis was carried out as for polymer 1, except that 90.0 g of the 25 wt % solution of K3 was added.

Polymer 3

The synthesis was carried out as for polymer 1, except that 140.0 g of the 25 wt % solution of K4 and 93.8 g of water were added.

Polymer 4

The synthesis was carried out as for polymer 1, except that 187.0 g of the 25 wt % solution of K2 and 93.8 g of water were added.

Polymer 5

The synthesis was carried out as for polymer 1, except that 117.0 g of the 25 wt % solution of K3 was added.

Polymer 6

The synthesis was carried out as for polymer 1, except that 187.2 of the 25 wt % solution of K4 and 93.8 g of water were added.

### Examples of the Start Temperature

Higher start temperatures lead to softer gels, since the molecular weights become lower. This could be prevented with a lower monomer concentration. In both cases, however, gels that can no longer be processed are formed. In general, therefore, start temperatures higher than 25° C. are not possible according to the inventive method, which includes size reduction of the gel and drying.

Polymer 7

The synthesis was carried out as described for polymer 2, but was started at 10° C.

Polymer 8

The synthesis was carried out as described for polymer 2, but was started at 15° C.

Polymer 9

The synthesis was carried out as described for polymer 2, but was started at 20° C.

Comparison Polymers:

Comparison Polymer 1

407.0 g of 50 wt % aqueous acrylamide solution was first placed in a polymerization vessel and 312.7 g of water as well as 0.15 g of Versenex 80 was mixed in. After the addition of 277.50 g of 60 wt % DIMAPA-quat, the pH was adjusted to 5.0 with 2 g of 50 wt % sulfuric acid and 0.30 g of formic acid, and the mixture was cooled to 0° C. and purged with nitrogen. After the addition of 0.40 g of ABAH (2,2′-azobis(2-methylpropionamidine)dihydrochloride), the polymerization was started with UV light. Within 25 minutes, the polymerization went from 0° C. to 80° C. The polymer was subjected to size reduction with a meat grinder and dried at 100° C. for 90 minutes. The product was ground to a particle-size fraction of 90 to 1400 μm.

Comparison Polymer 2

240.0 g of 50 wt % aqueous acrylamide solution was first placed in a polymerization vessel and 285.3 g of water as well as 210 mg of Versenex 80 was mixed in. After the addition of 466.7 g of 60 wt % DIMAPA-quat, the pH was adjusted to 5.0 with 8.0 g of 50 wt % sulfuric acid and 0.30 g of formic acid, and the mixture was cooled to 0° C. and purged with nitrogen. After the addition of 0.40 g of ABAH (2,2′-azobis(2-methylpropionamidine)dihydrochloride), the polymerization was started with UV light. Within 25 minutes, the polymerization went from 0° C. to 80° C. The polymer was subjected to size reduction with a meat grinder and dried at 100° C. for 90 minutes. The product was ground to a particle-size fraction of 90 to 1400 μm.

Comparison Polymer 3

342.0 g of 50 wt % aqueous acrylamide solution was first placed in a polymerization vessel and 394.7 g of water as well as 210 mg of Versenex 80 was mixed in. After the addition of 261.3 g of 80 wt % ADAME-quat, the pH was adjusted to 5.0 with 2.0 g of 50 wt % sulfuric acid, and the mixture was cooled to 0° C. and purged with nitrogen. After the addition of 0.40 g of ABAH (2,2′-azobis(2-methylpropionamidine)dihydrochloride), the polymerization was started with UV light. Within 25 minutes, the polymerization went from 0° C. to 80° C. The polymer was subjected to size reduction with a meat grinder and dried at 100° C. for 90 minutes. The product was ground to a particle-size fraction of 90 to 1400 μm.

Comparison Polymer 4

270.0 g of 50 wt % aqueous acrylamide solution was first placed in a polymerization vessel and 335.5 g of water as well as 210 mg of Versenex 80 was mixed in. After the addition of 393.8 g of 80 wt % ADAME-quat, the pH was adjusted to 5.0 with 2.0 g of 50 wt % sulfuric acid, and the mixture was cooled to 0° C. and purged with nitrogen. After the addition of 0.40 g of ABAH (2,2′-azobis(2-methylpropionamidine)dihydrochloride), the polymerization was started with UV light. Within 25 minutes, the polymerization went from 0° C. to 80° C. The polymer was subjected to size reduction with a meat grinder and dried at 100° C. for 90 minutes. The product was ground to a particle-size fraction of 90 to 1400 μm.

Comparison Polymer 5 (According to EP 262945 B1)

A mixture of 133.3 g of 75 wt % MADAME-quat solution, 250 g of K1 and 283.7 g of water was purged with nitrogen and heated to 70° C. After the addition of 3.0 mL of a 2% methanolic AIBN solution, the mixture was stirred for 3 hours at 70° C. (isothermally). The product viscosity was 19000 mPas.

Comparison Polymer 6 (According to EP 262945 B1)

The synthesis was carried out as in Comparison Example 5, except that 250.0 g of K1, 106.7 g of MADAME-quat, 40.0 g of acrylamide and 270.3 g of water were used.

Comparison Polymer 7 (According to EP 262945 B1)

The synthesis was carried out as in Comparison Example 5, except that 250.0 g of K1, 80.0 g of MADAME-quat, 80.0 g of acrylamide and 257.3 g of water were used.

Comparison Polymer 8 Start Temperature

The synthesis was carried out as in Comparison Example 6, but was started at 3° C. with 1000 ppm of Na₂S₂O₈, 7 ppm of FeSO₄ and 2000 ppm of Na₂S₂O₅. The temperature of the preparation rose to 33° C. in 24 minutes. Thereafter the mixture was stirred for another 60 minutes.

Comparison Polymer 9—Start Temperature

The synthesis was carried out as in Comparison Example 7, but was started at 3° C. with 500 ppm of Na₂S₂O₈, 7 ppm of FeSO₄ and 1000 ppm of Na₂S₂O₅. The temperature of the preparation rose to 31° C. in 40 minutes. Thereafter the mixture was stirred for another 60 minutes.

### APPLICATION EXAMPLES

The experiments on application were actually all performed on clarification sludge from liverich Clarifying Plant, but the sludge was sampled on different days and so the values can occasionally fluctuate for the same polymer/sludge combination. The same sludge batch was always used within a given example. As is known to those skilled in the art, the properties of the clarification sludge of one and the same clarifying plant can fluctuate with time.

### Application Example 1

Inventive polymers 1 to 3 were compared with comparison polymer 1 as well as with separate addition of second cationic polymer followed by first cationic polymer in the form of the comparison polymers without the second cationic polymer. The stirring time was 10 s and the filtrate volume was 200 mL.

AS: polymer quantity (“active substance”), DS: dry substance in the clarification sludge

Added quantity [kg
AS per metric
ton DS]

| | | |
|---|---|---|
| 3.9 | 4.2 | 4.5 |

Added quantity
[g AS per m³]

| | 120 | 130 | 140 |
|---|---|---|---|
| Comparison polymer 1 | 37 s | 22 s | 18 s |
| | 20 | 26 | 29 |
| Comparison polymer 1 with 10% K2 | 33 s | 24 s | 19 s |
| | 25 | 28 | 29 |
| Comparison polymer 1 with 10% K3 | 34 s | 21 s | 20 s |
| | 26 | 29 | 30 |
| Comparison polymer 1 with 10% K4 | 32 s | 18 s | 17 s |
| | 25 | 29 | 30 |
| Polymer 1 | 27 s | 18 s | 13 s |
| | 29 | 30 | 35 |
| Polymer 2 | 26 s | 15 s | 12 s |
| | 29 | 31 | 41 |
| Polymer 3 | 27 s | 15 s | 12 s |
| | 29 | 34 | 42 |

Data in s = time for 200 mL of filtrate, solution clear of fat

### Application Example 2

Inventive polymers 4 to 6 were compared with comparison polymer 2 as well as with separate addition of second cationic polymer followed by first cationic poilymer in the form of the comparison polymers without a proportion of the second cationic polymer.

The stirring time was 10 s and the filtrate volume was 200 mL.

AS: polymer quantity (“active substance”), DS: dry substance in the clarification sludge

Added quantity [kg
AS per metric
ton DS]

| | | |
|---|---|---|
| 4.2 | 4.5 | 4.8 |

Added quantity
[g AS per m3]

| | 130 | 140 | 150 |
|---|---|---|---|
| Comparison polymer 2 | 35 s | 25 s | 16 s |
| | 23 | 28 | 34 |
| Comparison polymer 2 with 10% K2 | 35 s | 25 s | 16 s |
| | 26 | 31 | 34 |
| Comparison polymer 2 with 10% K3 | 44 s | 28 s | 22 s |
| | 27 | 33 | 36 |
| Comparison polymer 2 with 10% K4 | 40 s | 31 s | 23 s |
| | 28 | 32 | 35 |
| Polymer 4 | 37 s | 19 s | 16 s |
| | 26 | 35 | 40 |
| Polymer 5 | 39 s | 25 s | 21 s |
| | 29 | 33 | 35 |
| Polymer 6 | 48 s | 35 s | 20 s |
| | 30 | 32 | 38 |

Data in s = time for 200 mL of filtrate, solution clear of fat

When rate of filtration and clarity of the filtrate are considered as the two parameters for efect, it is evident from the application examples that the inventive polymers have a better effect.

The products according to comparison examples V5 to V9 according to EP 262945 B1 were subject to application tests and were found to be much poorer than the inventive polymers. When added in quantities at which the inventive polymer yield good dewatering results, the comparison examples still do not achieve dewatering that even approximates satisfactory performance.

1. A powdery, water-soluble, cationic polymer composition comprising at least a first and a second cationic polymer, wherein the first and the second cationic polymers differ in molecular weight, wherein the first cationic polymer is formed by radical polymerization of its monomer constituents in the presence of the second cationic polymer in aqueous solution, wherein
the first and second cationic polymers comprise corresponding structural units, wherein
the polymerization of the first cationic polymer takes place in an aqueous solution of the second cationic polymer according to the method of adiabatic gel polymerization, and wherein
the ratio of the second to the first cationic polymer is from 0.01:10 to 1:3.

2. The composition to of claim 1, wherein the first cationic polymer has a weight-average molecular weight higher than 1 million.

3. The composition of to claim, wherein the second cationic polymer has a weight-average molecular weight lower than 1 million.

4. The composition to of claim 1, wherein the first and the second cationic polymer are formed using cationic monomers selected from the group consisting of cationized esters of (meth)acrylic acid and cationized amides of (meth)acrylic acid, in each case comprising a quaternized N atom.

5. The composition of claim 1, wherein the first or the second cationic polymer is produced by copolymerization with further water-soluble monomers.

6. The composition to of claim 1, wherein the first cationic polymer has a lower cationic charge density than the second cationic polymer.

7. The composition of claim 1, wherein the first cationic polymer comprises 20 to 90 wt % of cationic monomers.

8. The composition of claim 1, wherein the second cationic polymer comprises 70 to 100 wt % of cationic monomers.

9. A method for producing the cationic polymer composition of claim 1, comprising, subjecting the first cationic polymer to radical polymerization by adiabatic gel polymerization of its monomer constituents in the presence of the second cationic polymer in an aqueous solution to form the cationic polymer composition, wherein the ratio of the second to the first cationic polymer is from 0.01:10 to 1:3,
wherein
the aqueous solution comprises cationic and nonionic monomers, wherein the second cationic polymer is prepared with a concentration of 10 to 60 wt % of the composition, wherein the start temperature for the polymerization is adjusted to a range of −10° C. to 25° C., wherein oxygen is present and wherein the oxygen is purged by an inert gas,
wherein the polymerization reaction is exothermic, wherein the exothermic polymerization reaction of the monomers is started by addition of a polymerization initiator, wherein heating of the polymerization mixture takes place with formation of a polymer gel up to its maximum temperature and
wherein, after the maximum temperature has been reached, the polymer gel is subjected to mechanical size reduction and to drying.

10. The method of claim 9, wherein the start temperature of polymerization is adjusted to a range of 0° C. to 15° C.

11. The method to of claim 9, wherein the concentration of the aqueous solution of monomers and the second cationic polymer is 15 to 50 wt % of the composition.

12. The method to of claim 9, wherein the polymerization initiator comprises a redox system or a system that can be activated by UV radiation.

13. The method to of claim 9, wherein the polymerization is carried out on a polymerization belt.

14. The method to of claim 9, wherein after size reduction, the drying of the aqueous polymer gel is conducted at temperatures of 80° C. to 120° C. to thus obtain a moisture content of less than or equal to 12%.

15. A method of separating a solid from a liquid, comprising separating the solid from the liquid with the cationic polymer composition of claim 1.

16. A method for purifying wastewater or conditioning potable water, comprising purifying the wastewater or conditioning the potable water with the cationic polymer composition of claim 1.

17. A method of manufacturing paper comprising manufacturing the paper with the cationic polymer composition of claim 1.

18. The composition of claim 2, wherein the first cationic polymer has a lower cationic charge density than the second cationic polymer.

19. The composition of claim 3, wherein the first cationic polymer has a lower cationic charge density than the second cationic polymer.

20. The composition of claim 4, wherein the first cationic polymer has a lower cationic charge density than the second cationic polymer.
